(19) United States
(12) Patent Application Publication
(10) Pub. No.: US 2015/0351828 A1
(43) Pub. Date: Dec. 10, 2015

(54) SURGICAL INSTRUMENT INCLUDING RE-USABLE PORTION

(71) Applicant: COVIDIEN LP, Mansfield, MA (US)

(72) Inventor: AMARSINH D. JADHAV, ISLAMPUR (IN)

(21) Appl. No.: 14/611,569

(22) Filed: Feb. 2, 2015

Related U.S. Application Data
(60) Provisional application No. 62/008,354, filed on Jun. 5, 2014.

(51) Int. Cl. A61B 18/14

(52) U.S. Cl. CPC A61B 18/1445; A61B 2018/00178

(57) ABSTRACT

A surgical instrument includes a housing, a shaft assembly coupled to the housing, a drive sleeve disposed within the shaft assembly and reciprocatable therein, and a handle assembly operably associated with the housing. The shaft assembly includes a first shaft portion and a second shaft portion. The second shaft portion has first and second jaw members coupled to a distal end thereof. The reciprocatable drive sleeve includes a first drive-sleeve portion, slideably disposed at least partially within the first shaft portion, and a second drive-sleeve portion, slideably disposed at least partially within the second shaft portion. The surgical instrument also includes a shaft connector assembly configured to selectively couple the first shaft portion to the second shaft portion such that the first drive-sleeve portion is mechanically coupled to the second drive-sleeve portion.

### CROSS REFERENCE TO RELATED APPLICATION

The present application claims the benefit of and priority to U.S. Provisional Application Ser. No. 62/008,354, filed on Jun. 5, 2014, the entire contents of which are incorporated herein by reference.

### BACKGROUND

1. Technical Field

The present disclosure relates to surgical instruments. More particularly, the present disclosure relates to a surgical instrument, such as a vessel-sealing device, including a disposable portion configured to be selectively connectable to a re-usable portion.

2. Discussion of Related Art

Forceps utilize mechanical action to constrict, grasp, dissect and/or clamp tissue. By utilizing an electrosurgical forceps, a surgeon can utilize both mechanical clamping action and electrosurgical energy to effect hemostasis by heating the tissue and blood vessels to cauterize, coagulate/desiccate, seal and/or divide tissue. Bipolar electrosurgical forceps utilize two generally opposing electrodes that are operably associated with the inner opposing surfaces of end effectors and that are both electrically coupled to an electrosurgical generator. In bipolar forceps, the end-effector assembly generally includes opposing jaw assemblies pivotably mounted with respect to one another. In bipolar configuration, only the tissue grasped between the jaw assemblies is included in the electrical circuit. Because the return function is performed by one jaw assembly of the forceps, no patient return electrode is needed.

By utilizing an electrosurgical forceps, a surgeon can cauterize, coagulate/desiccate and/or seal tissue and/or simply reduce or slow bleeding by controlling the intensity, frequency and duration of the electrosurgical energy applied through the jaw assemblies to the tissue. During the sealing process, mechanical factors such as the pressure applied between opposing jaw assemblies and the gap distance between the electrically-conductive tissue-contacting surfaces (electrodes) of the jaw assemblies play a role in determining the resulting thickness of the sealed tissue and effectiveness of the seal.

A variety of types of end effectors have been employed for various types of surgery using a variety of types of surgical instruments, such as monopolar and bipolar surgical instruments, surgical instruments for applying fasteners, ultrasonic surgical instruments, and other surgical instruments including a shaft between an end effector and a handle portion. End effectors may engage tissue in a variety of ways to achieve a therapeutic and/or diagnostic effect, e.g., endocutter, grasper, cutter, stapler, clip applier, access device, drug/gene therapy delivery device, and energy-delivery device using ultrasound, radio frequency (RF), laser, etc.

Electrosurgical instruments, surgical cutting and fastening instruments, ultrasonic surgical instruments, and/or other types of surgical instruments including a shaft between an end effector and a handle portion may be constructed with modular parts such that parts can be readily replaced or otherwise changed by a user.

### SUMMARY

According to an aspect of the present disclosure, a surgical instrument is provided that includes a housing, a shaft assembly coupled to the housing, a drive sleeve disposed within the shaft assembly and reciprocatable therein, and a handle assembly operably associated with the housing. The shaft assembly includes a first shaft portion and a second shaft portion. The second shaft portion includes first and second jaw members coupled to a distal end thereof. The reciprocatable drive sleeve includes a first drive-sleeve portion, slideably disposed at least partially within the first shaft portion, and a second drive-sleeve portion, slideably disposed at least partially within the second shaft portion. The surgical instrument also includes a shaft connector assembly configured to mechanically couple the first shaft portion to the second shaft portion such that the first drive-sleeve portion is mechanically coupled to the second drive-sleeve portion.

According to another aspect of the present disclosure, a surgical instrument is provided that includes a reusable portion and a disposable portion. The reusable portion includes: a housing; a rotatable assembly coupled to the housing; a first shaft portion coupled to the rotatable assembly; a first knife-sleeve portion slideably disposed at least partially within the first shaft portion; a first drive-sleeve portion slideably disposed at least partially within the first knife-sleeve portion; a handle assembly operably associated with the housing and coupled to the first drive-sleeve portion; and a trigger assembly operably associated with the housing and coupled to the first knife-sleeve portion. The disposable portion includes: a second shaft portion having a proximal end and a distal end; an end-effector assembly coupled to the distal end of the second shaft portion; a second knife-sleeve portion slideably disposed at least partially within the second shaft portion; and a second drive-sleeve portion slideably disposed at least partially within the second knife-sleeve portion. The proximal end of the second shaft portion is configured to be selectively connectable to a distal end of the first shaft portion.

According to another aspect of the present disclosure, a method of assembling a surgical instrument includes: positioning a first shaft portion of a reusable portion of the surgical instrument relative to a longitudinal axis defined by a second shaft portion of a disposable portion of the surgical instrument. The reusable portion includes a housing, a rotatable assembly coupled to the housing, a first knife-sleeve portion slideably disposed at least partially within the first shaft portion, a first drive-sleeve portion slideably disposed at least partially within the first knife-sleeve portion, a handle assembly operably associated with the housing and coupled to the first drive-sleeve portion, and a trigger assembly operably associated with the housing and coupled to the first knife-sleeve portion. The disposable portion of the surgical instrument includes an end-effector assembly coupled to the distal end of the second shaft portion, a second knife-sleeve portion slideably disposed at least partially within the second shaft portion, and a second drive-sleeve portion slideably disposed at least partially within the second knife-sleeve portion. The method also includes positioning a coupling member within the second shaft portion for operably coupling the first knife-sleeve portion to the second knife-sleeve portion; and connecting the first drive-sleeve portion to the second drive-sleeve portion.

### BRIEF DESCRIPTION OF THE DRAWINGS

Objects and features of the presently-disclosed surgical instruments, such as vessel-sealing devices, including a disposable portion configured to be selectively connectable to a re-usable portion will become apparent to those of ordinary skill in the art when descriptions of various embodiments thereof are read with reference to the accompanying drawings, of which:

FIG. 1 is a perspective view of a surgical instrument, with parts separated, including a disposable portion configured to be selectively connectable to a re-usable portion in accordance with an illustrative embodiment of the present disclosure;

FIG. 2 is a perspective view of a surgical instrument, with parts separated, including another embodiment of a disposable portion configured to be selectively connectable to the re-usable portion shown in FIG. 1 in accordance with the present disclosure;

FIG. 3 is an enlarged, perspective view of the indicated area of detail of FIGS. 1 and 2 showing a sealing member associated with the disposable portion for forming a junction with the re-usable portion in accordance with an embodiment of the present disclosure;

FIG. 4 is an enlarged, perspective view of the indicated area of detail of FIG. 2 showing an end-effector assembly coupled to a shaft portion of the disposable portion in accordance with an embodiment of the present disclosure;

FIG. 5A is an enlarged, schematic view of the sealing member of FIGS. 3 and 4 in accordance with an embodiment of the present disclosure;

FIG. 5B is an enlarged, schematic view of another embodiment of a sealing member in accordance with the present disclosure;

FIG. 6 is a perspective view of portions of a surgical instrument, with parts separated, including a disposable portion, a re-usable portion, and a shaft connector assembly in accordance with an embodiment of the present disclosure;

FIG. 7 is a perspective view of parts of the disposable portion of the surgical instrument shown in FIG. 6 in accordance with an embodiment of the present disclosure;

FIG. 8 is an enlarged, schematic side view of the surgical instrument shown in FIG. 6 in accordance with an embodiment of the present disclosure;

FIG. 9 is an enlarged, cross-sectional view of the indicated area of detail of FIG. 6 showing a portion of the shaft connector assembly in accordance with an embodiment of the present disclosure;

FIG. 10 is an enlarged, cross-sectional view of the indicated area of detail of FIG. 6 showing a portion of the shaft connector assembly including a coupling member in accordance with an embodiment of the present disclosure;

FIG. 11 is an enlarged, cross-sectional view of the proximal end of the disposable portion of the surgical instrument shown in FIG. 6 including the coupling member shown in FIG. 9, with parts separated, in accordance with an embodiment of the present disclosure;

FIG. 12 is an enlarged, cross-sectional view of a shaft connector sleeve in accordance with an embodiment of the present disclosure;

FIG. 13 is an enlarged, cross-sectional view of a shaft portion in accordance with an embodiment of the present disclosure;

FIG. 14 is an enlarged, cross-sectional view of portions of a surgical instrument, with parts separated, including a disposable portion, a re-usable portion, and the shaft connector sleeve of FIG. 12 in accordance with an embodiment of the present disclosure; and

FIG. 15 is an enlarged, cross-sectional view of the assembled, portions of the surgical instrument of FIG. 14 in accordance with an embodiment of the present disclosure.

### DETAILED DESCRIPTION

Hereinafter, embodiments of a surgical instrument, such as a vessel-sealing device, including a disposable portion configured to be selectively connectable to a re-usable portion of the present disclosure are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the apparatus, or component thereof, closer to the user and the term “distal” refers to that portion of the apparatus, or component thereof, farther from the user.

This description may use the phrases “in an embodiment,” “in embodiments,” “in some embodiments,” or “in other embodiments,” which may each refer to one or more of the same or different embodiments in accordance with the present disclosure.

Various embodiments of the present disclosure provide surgical instruments including a re-usable portion and a disposable portion configured to be selectively connectable to the re-usable portion. Various embodiments of the present disclosure provide surgical instruments suitable for sealing, cauterizing, coagulating, desiccating, cutting, and/or dissecting vessels and vascular tissue. Embodiments of the presently-disclosed surgical instrument may be suitable for utilization in endoscopic surgical procedures and/or suitable for utilization in open surgical applications.

Embodiments of the presently-disclosed surgical instruments may be implemented using a variety of types of energy, e.g., electrosurgical energy at radio frequencies (RF) or at other frequencies, ultrasonic, optical, and/or thermal energy. Embodiments of the presently-disclosed surgical instruments may be connected through a suitable cable to a generator and/or other suitable power source. Although the following description describes the use of a vessel-sealing device, the teachings of the present disclosure may also apply to a variety of surgical devices with an end-effector assembly and including a shaft, a handle assembly, and other components which mutually cooperate to impart movement to one or more components of the end-effector assembly. The teachings of the present disclosure may also apply to surgical devices with an end-effector assembly configured to apply surgical fasteners.

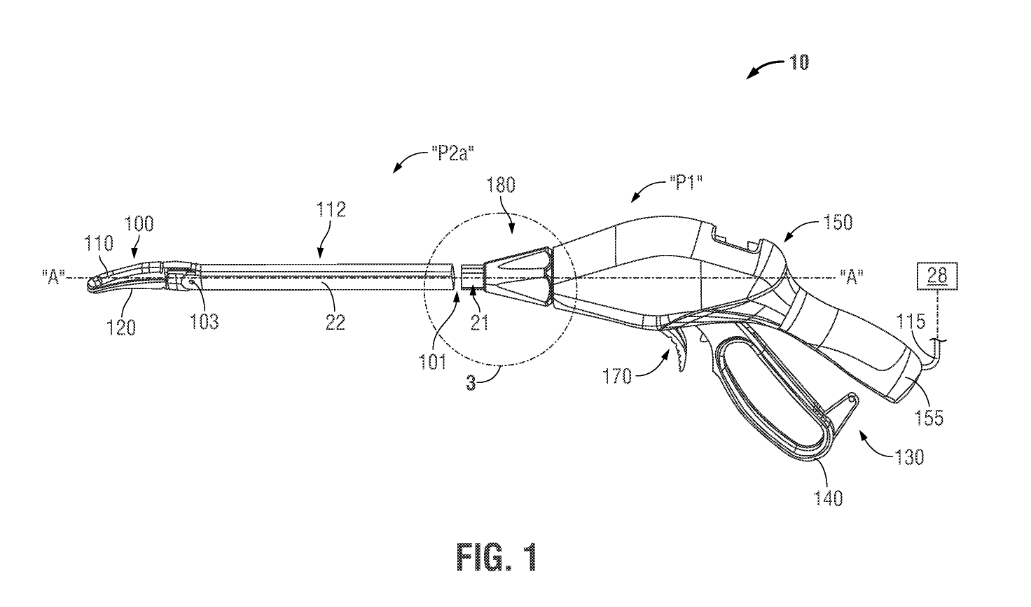

In FIG. 1, an embodiment of a surgical instrument 10 including a re-usable portion “P1” and a disposable portion “P2a” is shown for use with various surgical procedures. Surgical instrument 10 generally includes a housing 150, a handle assembly 130, a rotating assembly 180, a trigger assembly 170, a shaft assembly 112, and an end-effector assembly 100. End-effector assembly 100 generally includes two jaw members 110 and 120 disposed in opposing relation relative to one another. Shaft assembly 112 extends distally from the housing 150 and includes a first shaft portion 21 and a second shaft portion 22. Surgical instrument 10 may include additional, fewer, or different components than shown in FIG. 1, depending upon a particular purpose or to achieve a desired result. Surgical instrument 10 may include any feature or combination of features of the shaft connector assembly embodiments disclosed herein.

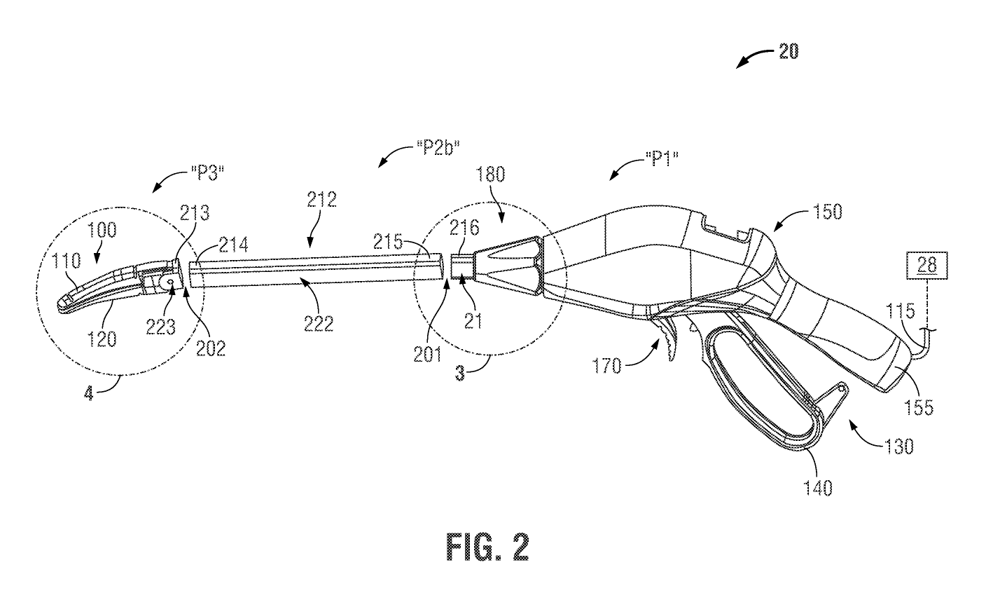

In some embodiments, as shown for example in FIG. 1, the re-usable portion “P1” includes the housing 150, the handle assembly 130, the rotating assembly 180, the trigger assembly 170, and the first shaft portion 21. Disposable portion “P2a” includes the second shaft portion 22 of the shaft assembly 112 and the end-effector assembly 100, which is disposed at the distal end of the second shaft portion 22. In some embodiments, the end-effector assembly 100 is selectively connectable to the distal end of the second shaft portion 22. The shape and size of the first shaft portion 21 and the second shaft portion 22 may be varied from the configuration depicted in FIG. 1. Although the shaft assembly 112 shown in FIG. 1 includes two shaft portions, it is to be understood that any number of shaft portions (e.g., three shaft portions shown in FIG. 2) may be utilized.

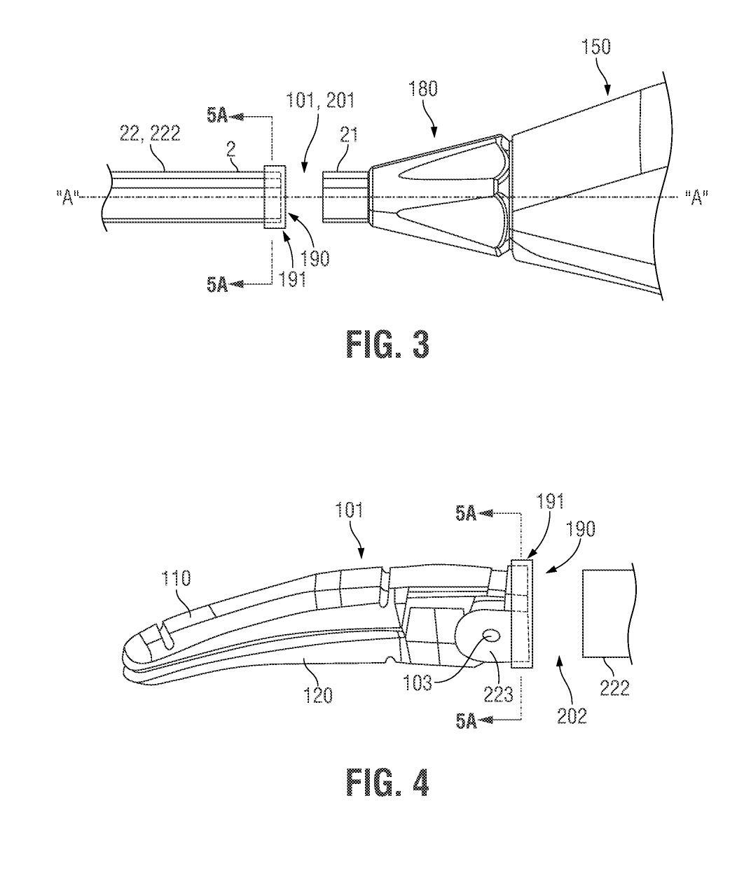

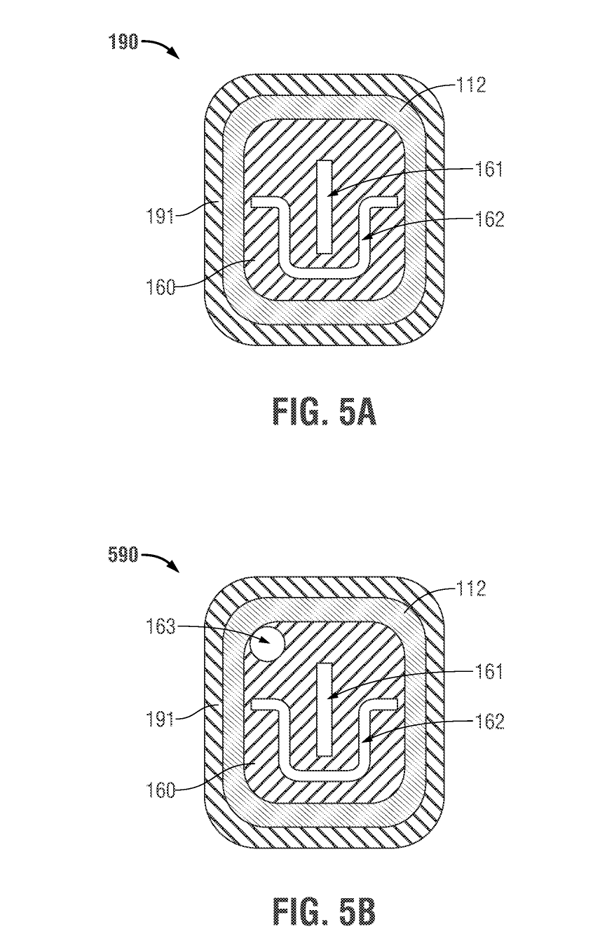

In some embodiments, as shown for example in FIG. 3, surgical instrument 10 includes a resilient sealing member 190 disposed at the connecting junction 101 between the first shaft portion 21 and the second shaft portion 22. In some embodiments, sealing member 190 is positioned in sealing contact with the proximal end of the second shaft portion 22 and configured to sealably join the first shaft portion 21 and the second shaft portion 22 when connected to one another. Sealing member 190 includes a flange portion 191 including an inner peripheral wall configured to sealably engage the outer surface 2 of the second shaft portion 22 of the shaft assembly 112. Sealing member 190 may additionally, or alternatively, be positioned in sealing contact with the distal end of the first shaft portion 21. Sealing member 190 is described in more detail below with reference to FIG. 5A.

As shown in FIGS. 1 and 3, the rotatable assembly 180 is operatively associated with the housing 150 and is rotatable in either direction about a longitudinal axis “A-A” defined by the shaft assembly 112. The longitudinal axis “A-A” extends through the first and second shaft portions 11 and 12, respectively, when coupled to one another. End-effector assembly 100 is rotatable about the longitudinal axis “A-A” through rotation, either manually or otherwise, of the rotatable assembly 180. One or more components of the surgical instrument 10, e.g., the housing 150, the rotation knob 180, the trigger assembly 170, and/or the end-effector assembly 100, may be adapted to mutually cooperate to grasp, seal and/or divide tissue, e.g., tubular vessels and vascular tissue.

End-effector assembly 100 may be configured as a unilateral assembly, i.e., the end-effector assembly 100 may include a stationary or fixed jaw member, e.g., jaw member 120, mounted in fixed relation to the second shaft portion 22, and a moveable jaw member, e.g., jaw member 110, mounted about a pivot pin 103 coupled to the fixed jaw member. Alternatively, the surgical instrument 10 may include a bilateral assembly, i.e., both jaw members 110 and 120 are moveable relative to one another. Jaw members 110 and 120 may be curved at various angles to facilitate manipulation of tissue and/or to provide enhanced line-of-sight for accessing targeted tissues. It is to be understood that a variety of different end-effector assemblies may be utilized with the surgical instrument 10.

In some embodiments, the surgical instrument 10 includes a cable assembly 115. Cable assembly 115 may be formed from a suitable flexible, semi-rigid or rigid cable, and may connect directly to an electrosurgical power generating source 28. Electrosurgical power generating source 28 may be any generator suitable for use with electrosurgical devices, and may be configured to provide various frequencies of electromagnetic energy. Surgical instrument 10 may alternatively be configured as a battery-powered wireless instrument.

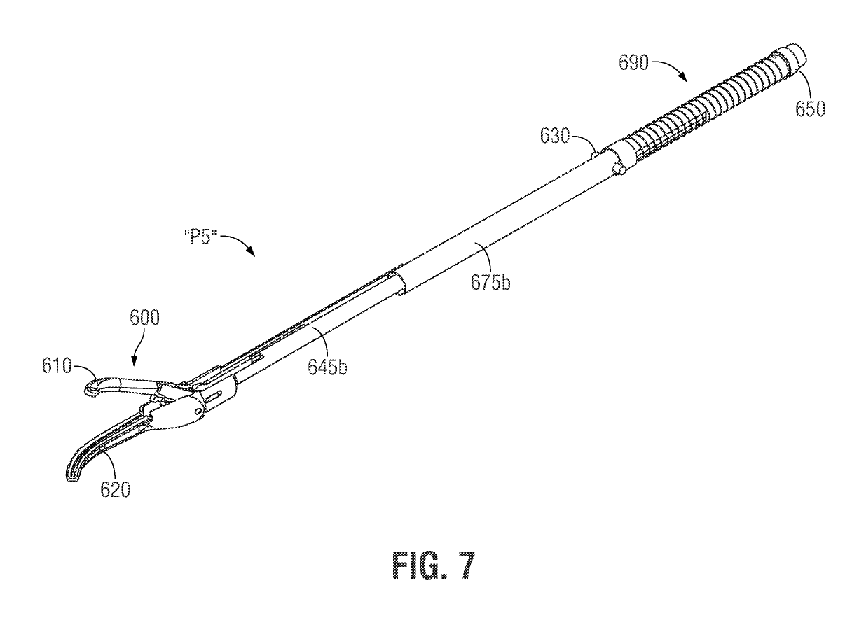

Handle assembly 130 includes a fixed handle 155 and a movable handle 140. Movable handle 140 of the handle assembly 130 is ultimately connected to the drive assembly (not explicitly shown). As can be appreciated, applying force to move the movable handle 140 toward the fixed handle 155 pulls a drive sleeve (e.g., second drive-sleeve portion 645b shown in FIG. 7) proximally to impart movement to the end-effector assembly 100, e.g., to effect movement of the jaw members 110 and 120 from an open position, wherein the jaw members 110 and 120 are disposed in spaced relation relative to one another, to a clamping or closed position, wherein the jaw members 110 and 120 cooperate to grasp tissue therebetween. Examples of handle assembly embodiments of the surgical instrument 10 are described in commonly-assigned U.S. Pat. No. 7,150,097 entitled “Method Of Manufacturing Jaw Assembly For Vessel Sealer And Divider,” commonly-assigned U.S. Pat. No. 7,156,846 entitled “Vessel Sealer And Divider For Use With Small Trocars And Cannulas,” commonly-assigned U.S. Pat. No. 7,597,693 entitled “Vessel Sealer And Divider For Use With Small Trocars And Cannulas” and commonly-assigned U.S. Pat. No. 7,771,425 entitled “Vessel Sealer And Divider Having A Variable Jaw Clamping Mechanism.”

FIG. 2 shows an embodiment of a surgical instrument 20 that includes a disposable portion “P2b” and a disposable portion “P3” including the end-effector assembly 100 of the surgical instrument 10 of FIG. 1. Although the end-effector assembly 100 is shown, it is to be understood that other end-effector assembly configurations may be used, which may include additional, fewer, or different components than shown in FIG. 2. Surgical instrument 20 includes the re-usable portion “P1” including the housing 150, the handle assembly 130, the rotating assembly 180, and the trigger assembly 170 of the surgical instrument 10. Surgical instrument 20 generally includes a shaft assembly 212 disposed between the end-effector assembly 100 and the handle assembly 150. Surgical instrument 20 may include additional, fewer, or different components than shown in FIG. 2, depending upon a particular purpose or to achieve a desired result. It is to be understood that a variety of different end-effector assemblies may be utilized with the surgical instrument 20.

In some embodiments, as shown for example in FIG. 2, shaft assembly 212 includes the first shaft portion 21, a second shaft portion 222, and a third shaft portion 223. Disposable portion “P2b” includes the second shaft portion 222. Disposable portion “P3” includes the third shaft portion 223 and the end-effector assembly 100. Second shaft portion 222 may be sterilized after usage, in which the portion “P2b” may be re-usable. Second shaft portion 222 includes a distal end 214 and a proximal end 215, wherein the proximal end 215 of the second shaft portion 222 is configured to be selectively connectable to the distal end 216 of the first shaft portion 21, and the distal end 214 of the second shaft portion 222 is configured to be selectively connectable to the proximal end 213 of the third shaft portion 223.

As shown in FIGS. 3 and 4, a resilient sealing member 190 is disposed at the connecting junction 201 (FIG. 3) between the first shaft portion 21 and the second shaft portion 222, and another resilient sealing member 190 is disposed at the connecting junction 202 (FIG. 4) between the second shaft portion 222 and the third shaft portion 223.

In FIG. 5A, the resilient sealing member 190 is shown, in cross-section, and includes the flange portion 191, as shown for example in FIG. 3, and a wall portion 160. Flange portion 191 and the wall portion 160 may be formed of any suitable material or combination of materials. In some embodiments, the flange portion 191 is formed of an elastomeric material, and the wall portion 160 may be formed of a rigid or semi-rigid material. Wall portion 160 may be formed of a lubricous plastic material.

As shown in FIG. 5A, the wall portion 160 generally includes a first opening 161 and a second opening 162 defined therethrough. In some embodiments, the first opening 161 and the second opening 162 are configured to sealingly engage one or more moveable components that longitudinally extend through the shaft assembly (e.g., shaft assembly 112 shown in FIG. 1, or shaft assembly 212 shown in FIG. 2). The shape and size of the first opening 161 and the second opening 162 may vary from the configuration depicted in FIG. 5A. Although the wall portion 160 shown in FIG. 5A includes two openings, it is to be understood that any number of suitable openings (e.g., three openings shown in FIG. 5B) may be utilized.

FIG. 5B shows a resilient sealing member 590, which is similar to resilient sealing member 190 shown in FIG. 5A, except for the third opening 163 in the wall member 160 shown in FIG. 5B. Further description of like elements is omitted in the interests of brevity. In some embodiments, the third opening 163 is configured to receive a wire or a plurality of wires (e.g., wiring bundle) therethrough. The shape, size, and position of the third opening 163, e.g., in relation to the first opening 161 and the second opening 162, may be varied from the configuration depicted in FIG. 5B.

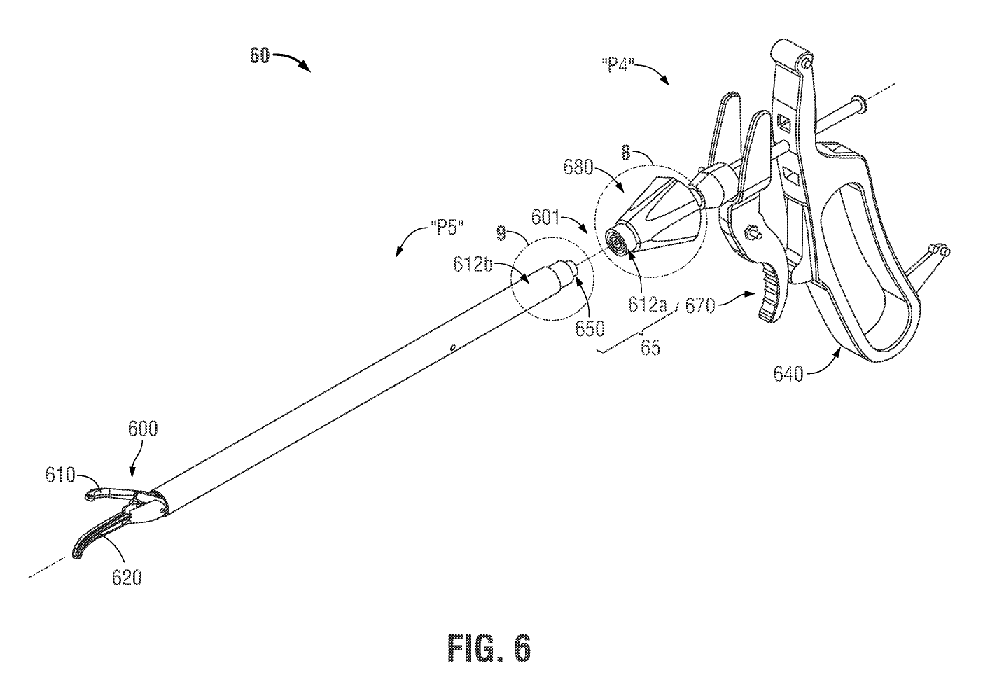

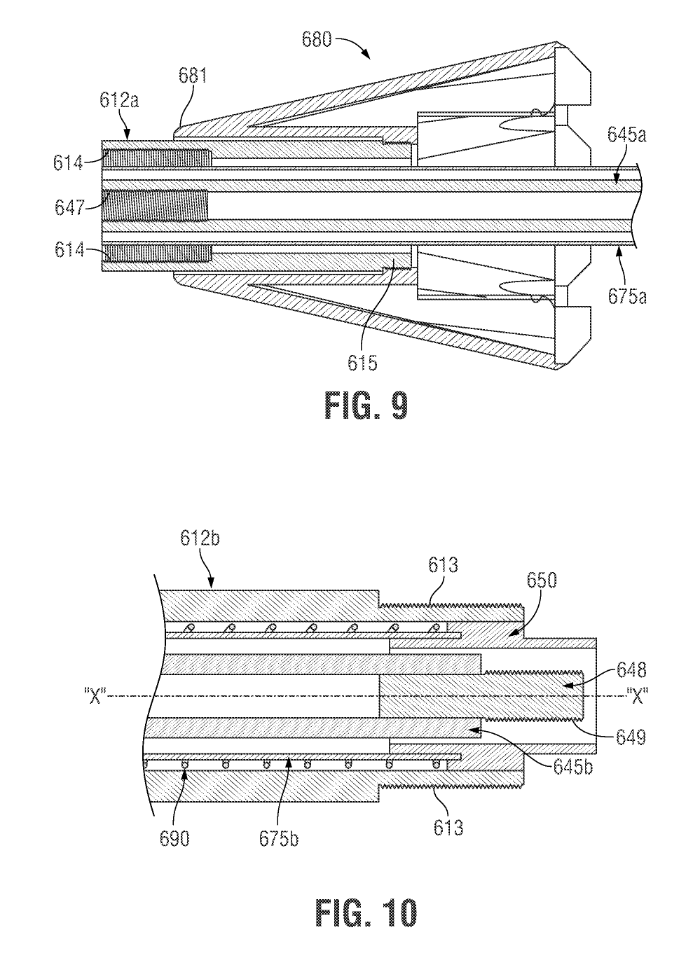

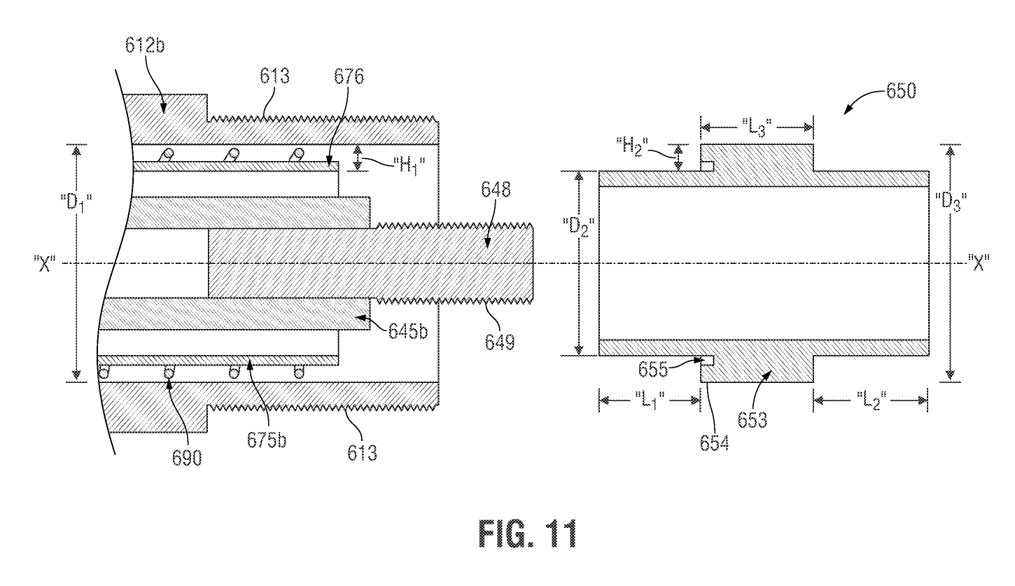

FIGS. 6 through 8 show parts of a surgical instrument 60 that includes a re-usable portion “P4,” a disposable portion “P5,” and a shaft connector assembly (shown generally as 65 in FIG. 6) configured to allow the disposable portion “P5” to be selectively connected to the re-usable portion “P4.” In some embodiments, the re-usable portion “P4” includes a first shaft portion 612a, a rotatable assembly 680, a trigger assembly 670, a handle assembly 640, and a housing (not shown in FIG. 6). The disposable portion “P5” includes a second shaft portion 612b and an end-effector assembly 600 disposed at the distal end of the second shaft portion 612b. In some embodiments, shown for example in FIGS. 9 and 10, surgical instrument 60 additionally includes first and second drive-sleeve portions 645a and 645b, respectively, and first and second knife-sleeve portions 675a and 675b, respectively, associated with the first and second shaft portions 612a and 612b, respectively.

End-effector assembly 600 generally includes two jaw members 610 and 620 disposed in opposing relation relative to one another. In some embodiments, end-effector assembly 600 may be selectively connectable to the distal end of the second shaft portion 612b. Referring to FIGS. 6 through 8, the handle assembly 640, the first drive-sleeve portion 645a, and the second drive-sleeve portion 645b mechanically communicate to impart movement to at least one of the jaw members 610 and 620, and the trigger assembly 670 and the first knife-sleeve portion 675a mechanically communicate to impart movement to a knife (not shown). Rotatable assembly 680, the trigger assembly 670, the handle assembly 640, and the end-effector assembly 600 shown in FIG. 6 are similar to the rotatable assembly 180, the trigger assembly 170, the handle assembly 140, and the end-effector assembly 100 shown in FIG. 1 and further description thereof is omitted in the interests of brevity.

Shaft connector assembly 65 includes the proximal end of the first shaft portion 612a, the distal end of the second shaft portion 612b, and a coupling member 650. In some embodiments, the proximal end of the first shaft portion 612a is configured to threadedly couple to the distal end of the second shaft portion 612b. Components of the shaft connector assembly 65 are shown in more detail in FIGS. 9 through 11.

In FIG. 7, the disposable portion “P5” of the surgical instrument 60 is shown (second shaft portion 612b is omitted in FIG. 7) and includes the end-effector assembly 600, the second drive-sleeve portion 645b, and the second knife-sleeve portion 675b. In some embodiments, a compression spring 690 is provided for biasing the second knife-sleeve portion 675b, and a connection pin 630 may be provided to provide a base surface for spring compression. Pin 630 may be configured for sliding connection with the second knife-sleeve portion 675b and the second drive-sleeve portion 645b. In some embodiments, the pin 630 may be coupled to the second shaft portion 612b.

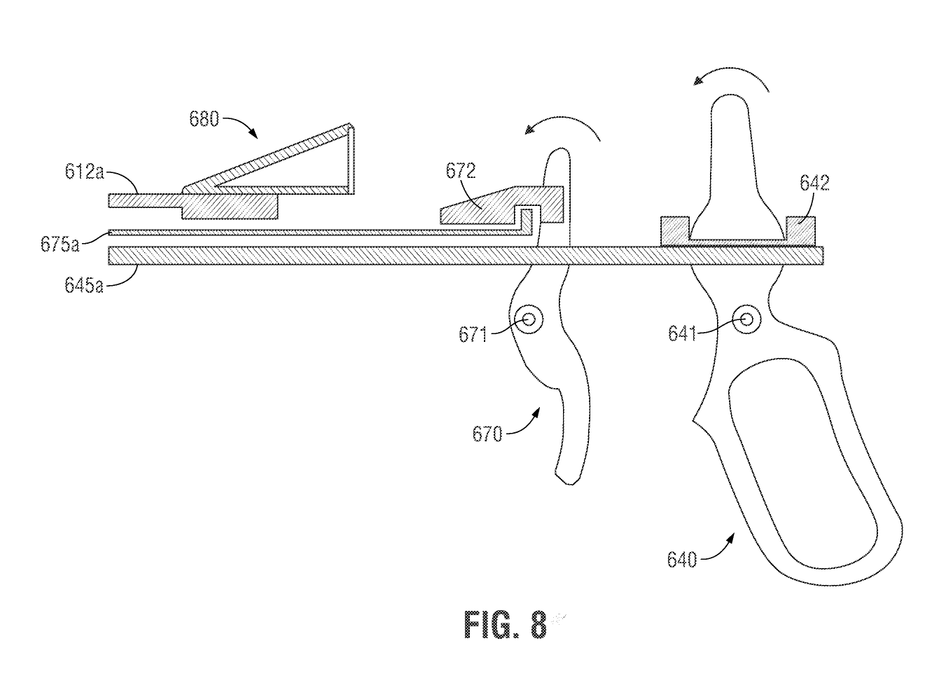

Referring to FIG. 8, handle assembly 640 includes a pivot 641 and is coupled via a bushing 642 to the first drive-sleeve portion 645a, and the trigger assembly 670 includes a pivot 671 and is coupled via a bushing 672 to the first knife-sleeve portion 675a. Applying force to move the handle member 640 between a first position and a second position moves the first drive-sleeve portion 645a in an axial direction. Applying force to move the trigger assembly 670 toward the handle member 640 pulls the knife sleeve 675a proximally.

FIG. 9 shows, in cross section, the rotatable assembly 680, the first shaft portion 612a, and the first drive-sleeve portion 645a and the first knife-sleeve portion 675a partially disposed within the first shaft portion 612a. First shaft portion 612a is connected to the rotatable assembly 680 such that rotation of the rotatable assembly 680 causes rotation of the first shaft portion 612a. In some embodiments, the first shaft portion 612a has its proximal end 615 threadedly or otherwise fixedly connected to the rotatable assembly 680. First shaft portion 612a may additionally, or alternatively, be adhesively and/or mechanically coupled to the rotatable assembly 680. In other embodiments, the first shaft portion 612a may be removeably coupled to the rotatable assembly 680.

In some embodiments, as shown for example in FIG. 9, a distal portion of the first shaft portion 612a extends outwardly from the distal end 681 of the rotatable assembly 680, e.g., to facilitate ease of connection between the disposable portion “P5” of the surgical instrument 60 and the re-usable portion “P4.” As shown in FIG. 9, first shaft portion 612a includes a threaded portion 614 at the distal end of the first shaft portion 612a, and the first drive-sleeve portion 645a includes a threaded portion 647 at the distal end of the drive-sleeve portion 645a. In some embodiments, shown for example in FIGS. 9 and 10, the first drive-sleeve portion 645a is disposed within the first knife-sleeve portion 675a, and the second drive-sleeve portion 645b is disposed within the second knife-sleeve portion 675b.

Referring to FIGS. 10 and 11, the proximal end of the second shaft portion 612b is shown, in cross section, and includes a threaded portion 613 configured to engage the threaded portion 614 of the first shaft portion 612a when connected to one another. As shown in FIGS. 10 and 11, the shaft connector assembly 65 (FIG. 6) includes a rod member 648 coupled to the distal end of the second drive-sleeve portion 645b and extending proximally outward therefrom. Rod member 648 includes a threaded portion 649 configured to engage the threaded portion 647 (FIG. 9) of the first drive-sleeve portion 645a when connected to one another.

As shown in FIG. 11, the coupling member 650 includes a substantially cylindrical body having an outer diameter “D2” and an engagement member 653 having an outer diameter “D3.” In some embodiments, the diameter “D3” of the engagement member 653 is substantially equal to the inner diameter “Di” of the second shaft portion 612b. In some embodiments, the height “H2” of the engagement member 653 is substantially equal to the height “H1” of the space within the second shaft portion 612b occupied by the compression spring 690 and the second knife-sleeve portion 675b.

Coupling member 650 is configured to operably couple the first knife-sleeve portion 675a (FIG. 9) to the second knife-sleeve portion 675b, and may be fixedly connected to the second knife-sleeve portion 675b. Engagement member 653 may have any suitable length “L3,” and the coupling member 650 may have any suitable length “L₁”+“L₂”+“L₃.” In some embodiments, as shown for example in FIG. 11, the distal end 654 of the coupling member 150 includes an annular recess 655 configured to receive a proximal end 676 of the second knife-sleeve portion 675b therein, e.g., to enhance stability and/or ease of assembly.

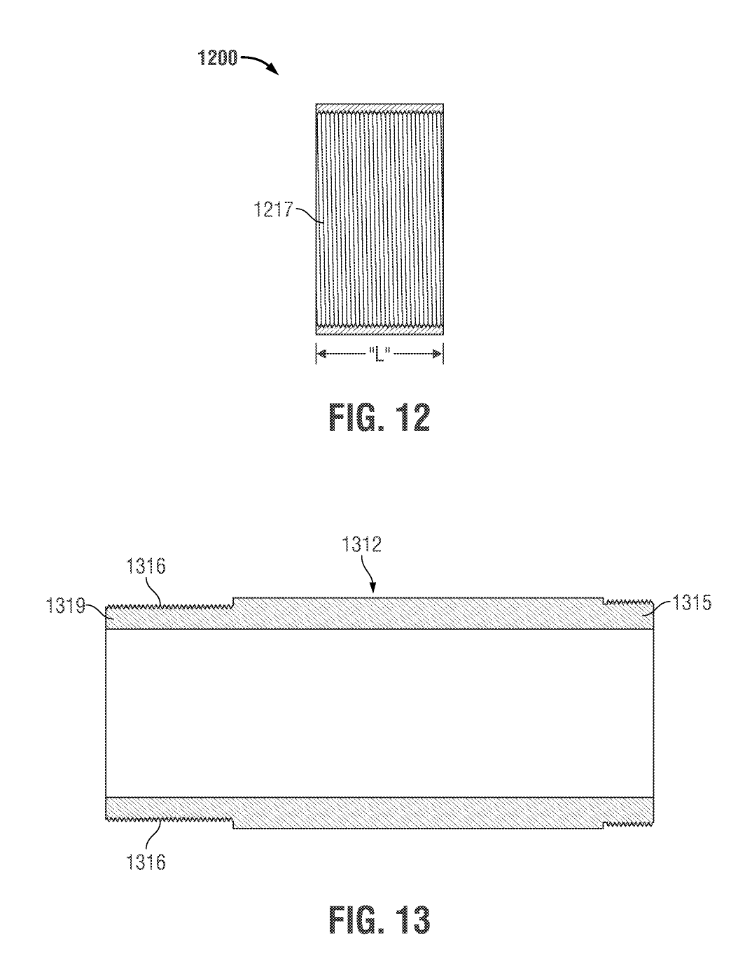

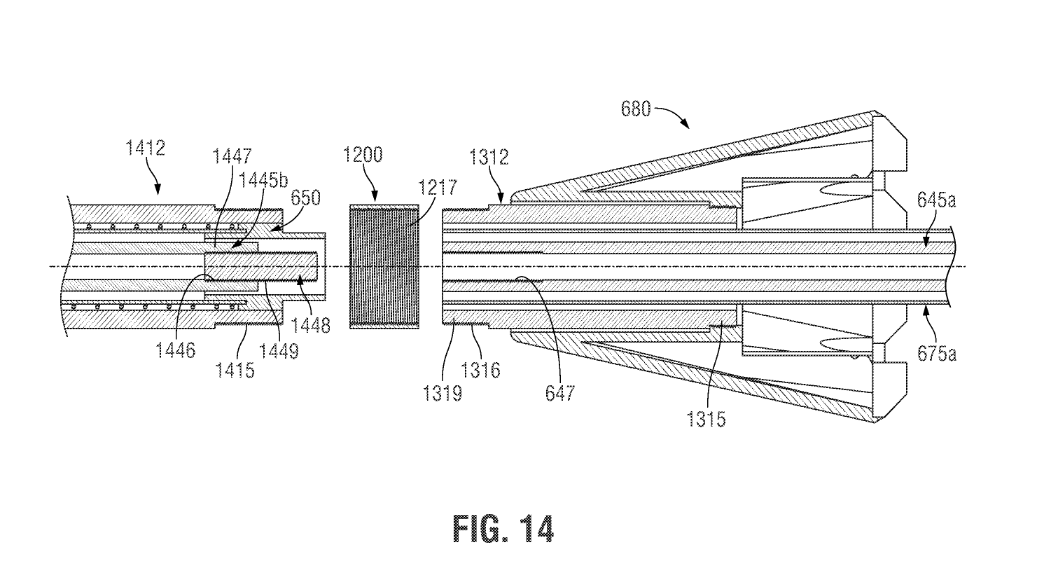

FIG. 12 shows a shaft connector sleeve 1200 including internal threads 1217 and configured for threaded engagement with the threaded surfaces of a first shaft portion 1312 (FIG. 13) and a second shaft portion 1412 (shown in FIG. 14) of a surgical instrument (e.g., surgical instrument 60 shown in FIG. 6). Shaft connector sleeve 1200 may have any suitable length “L.”

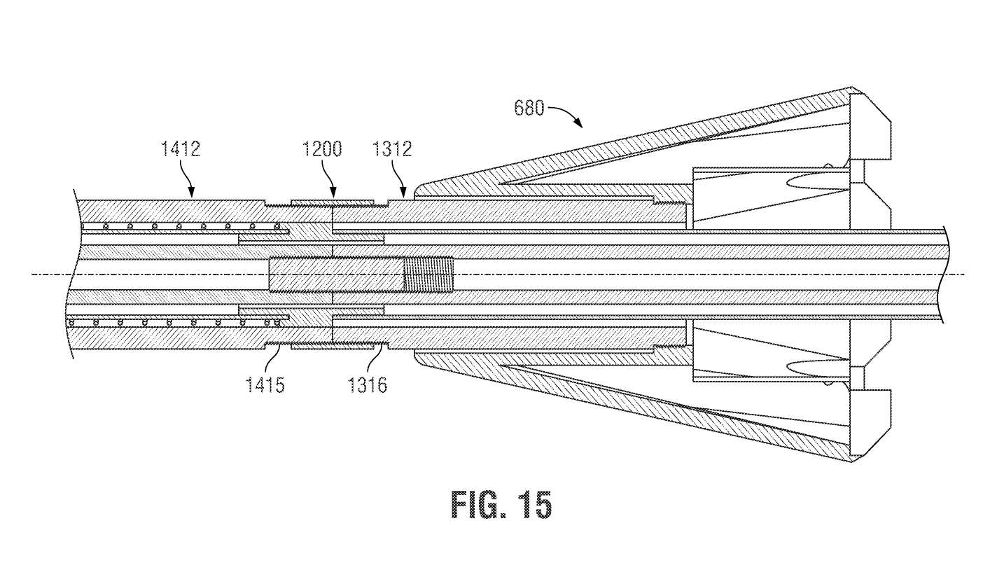

FIG. 13 shows a first shaft portion 1312 having a distal end 1319 and a proximal end 1315. First shaft portion 1312 is configured for connection to the rotatable assembly 680 such that rotation of the rotatable assembly 680 causes rotation of the first shaft portion 1312. In some embodiments, the first shaft portion 1312 has its proximal end 1315 (FIGS. 14 and 15) threadedly or otherwise fixedly connected to the rotatable assembly 680. First shaft portion 1312 may additionally, or alternatively, be adhesively and/or mechanically coupled to the rotatable assembly 680. In other embodiments, the first shaft portion 1312 may be removeably coupled to the rotatable assembly 680.

First shaft portion 1312 is provided with threads 1316 at its distal end 1319 configured for engagement with internal threads 1217 of the shaft connector sleeve 1200 (FIG. 12). First shaft portion 1312 is similar to the first shaft portion 612a shown in FIG. 10, except for the external threads 1316 shown in FIGS. 13 through 15. Further description of like elements is omitted in the interests of brevity.

FIG. 14 shows a second shaft portion 1412 including threads 1415 configured for engagement with threads 1217 of the shaft connector sleeve 1200. As shown in FIG. 14, a first knife-sleeve portion 675a is slideably disposed at least partially within the first shaft portion 1312, and a first drive-sleeve portion 645a is slideably disposed at least partially within the first knife-sleeve portion 675a.

A second drive-sleeve portion 1445b is slideably disposed at least partially within the second shaft portion 1412. A rod member 1448 is coupled to the distal end of a second drive-sleeve portion 1445b and extends proximally outward therefrom. Rod member 1448 includes a threaded portion 1449 configured to engage the threaded portion 647 of the first drive-sleeve portion 645a when connected to one another.

Second shaft portion 1412 is similar to the second shaft portion 612b shown in FIG. 10, except for the different configurations of the threads 613 shown in FIG. 10 and the threads 1415 shown in FIGS. 13 through 15. Second drive-sleeve portion 1445b and the rod member 1448 are similar to the second drive-sleeve portion 645b and the rod member 648, respectively, shown in FIG. 10, except for the threads 1446 at the proximal end 1447 of the second drive-sleeve portion 1445b and the configuration of the threads 1449 of the rod member 1448 shown in FIG. 14. Further description of like elements is omitted in the interests of brevity.

FIG. 15 shows the first shaft portion 1312 and the second shaft portion 1412 mechanically coupled to one another via the shaft connector sleeve 1200.

A method of assembling a surgical instrument in accordance with the present disclosure includes positioning a first shaft portion 612a of a reusable portion “P4” of the surgical instrument 60 relative to a longitudinal axis “A-A” defined by a second shaft portion 612b of a disposable portion “P4” of the surgical instrument 60. The reusable portion “P4” includes a housing, a rotatable assembly 680 coupled to the housing, a first knife-sleeve portion slideably disposed at least partially within the first shaft portion 612a, a first drive-sleeve portion 645a slideably disposed at least partially within the first knife-sleeve portion 675a, a handle assembly 640 operably associated with the housing and coupled to the first drive-sleeve portion 645a, and a trigger assembly 670 operably associated with the housing and coupled to the first knife-sleeve portion 645a. The disposable portion “P5” of the surgical instrument 60 includes an end-effector assembly 600 coupled to the distal end of the second shaft portion 612b, a second knife-sleeve portion 675b slideably disposed at least partially within the second shaft portion 612b, and a second drive-sleeve portion 645b slideably disposed at least partially within the second knife-sleeve portion 675b. The method also includes: positioning a coupling member 650 within the second shaft portion 612b for operably coupling the first knife-sleeve portion 675a to the second knife-sleeve portion 675b; and threadedly connecting the first drive-sleeve portion 645a to the second drive-sleeve portion 645b.

In some embodiments, positioning the coupling member 650 within the second shaft portion 612b includes fixedly connecting a proximal end of the second knife-sleeve portion 675b within an annular recess 655 formed in the coupling member 650.

The above-described surgical instrument embodiments include a re-usable portion and a disposable portion configured to be selectively connectable to the re-usable portion and may be suitable for use in a variety of procedures and operations. The above-described surgical instrument embodiments may be suitable for utilization with endoscopic surgical procedures and/or hand-assisted, endoscopic and laparoscopic surgical procedures and/or open surgical applications.

The above-described surgical instrument embodiments may also be configured to work with robotic surgical systems and what is commonly referred to as “Telesurgery.” Such systems employ various robotic elements to assist the surgeon in the operating theater and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include, remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

Although embodiments have been described in detail with reference to the accompanying drawings for the purpose of illustration and description, it is to be understood that the disclosed apparatus are not to be construed as limited thereby. It will be apparent to those of ordinary skill in the art that various modifications to the foregoing embodiments may be made without departing from the scope of the disclosure.

What is claimed is:

1. A surgical instrument, comprising:
a housing;
a shaft assembly coupled to the housing, the shaft assembly including a first shaft portion and a second shaft portion, the second shaft portion having first and second jaw members coupled to a distal end thereof;
a drive sleeve disposed within the shaft assembly and reciprocatable therein, the drive sleeve including:
a first drive-sleeve portion slideably disposed at least partially within the first shaft portion; and
a second drive-sleeve portion slideably disposed at least partially within the second shaft portion;
a handle assembly operably associated with the housing; and
a shaft connector assembly configured to selectively couple the first shaft portion to the second shaft portion such that the first drive-sleeve portion is mechanically coupled to the second drive-sleeve portion.

2. The surgical instrument of claim 1, wherein the second shaft portion is disposable.

3. The surgical instrument of claim 1, wherein the shaft connector assembly includes a rod member coupled to a distal end of the second drive-sleeve portion and extending proximally.

4. The surgical instrument of claim 3, wherein the rod member includes a threaded portion configured to engage a threaded portion of the first drive-sleeve portion when connected to one another.

5. The surgical instrument of claim 1, further comprising a trigger assembly operably associated with the housing.

6. The surgical instrument of claim 5, further comprising a knife sleeve including a first knife-sleeve portion and a second knife-sleeve portion.

7. The surgical instrument of claim 6, wherein the trigger assembly is coupled to the first knife-sleeve portion.

8. The surgical instrument of claim 5, wherein the shaft connector assembly includes a coupling member disposed within the second shaft portion and fixedly connected to the second knife-sleeve portion.

9. The surgical instrument of claim 8, wherein the coupling member includes an annular recess configured to receive a proximal end of the second knife-sleeve portion therein.

10. A surgical instrument, comprising:
a reusable portion, including:
a housing;
a rotatable assembly coupled to the housing;
a first shaft portion coupled to the rotatable assembly;
a first knife-sleeve portion slideably disposed at least partially within the first shaft portion;
a first drive-sleeve portion slideably disposed at least partially within the first knife-sleeve portion;
a handle assembly operably associated with the housing and coupled to the first drive-sleeve portion; and
a trigger assembly operably associated with the housing and coupled to the first knife-sleeve portion; and
a disposable portion, including:
a second shaft portion having a proximal end and a distal end, the proximal end configured to be selectively connectable to a distal end of the first shaft portion;
an end-effector assembly coupled to the distal end of the second shaft portion;
a second knife-sleeve portion slideably disposed at least partially within the second shaft portion; and
a second drive-sleeve portion slideably disposed at least partially within the second knife-sleeve portion.

11. The surgical instrument of claim 10, wherein end-effector assembly includes two jaw members, at least one of the jaw members movable relative to the other from a first position wherein the jaw members are disposed in spaced relation relative to one another to at least a second position closer to one another wherein the jaw members cooperate to grasp tissue therebetween.

12. The surgical instrument of claim 10, further comprising a coupling member disposed within the second shaft portion and configured to operably couple the first knife-sleeve portion to the second knife-sleeve portion.

13. The surgical instrument of claim 12, wherein the coupling member is fixedly connected to the second knife-sleeve portion.

14. The surgical instrument of claim 13, wherein the coupling member includes an annular recess configured to receive a proximal end of the second knife-sleeve portion therein.

15. A method of assembling a surgical instrument, comprising:
positioning a first shaft portion of a reusable portion of the surgical instrument relative to a longitudinal axis defined by a second shaft portion of a disposable portion of the surgical instrument,
the reusable portion including a housing, a rotatable assembly coupled to the housing, a first knife-sleeve portion slideably disposed at least partially within the first shaft portion, a first drive-sleeve portion slideably disposed at least partially within the first knife-sleeve portion, a handle assembly operably associated with the housing and coupled to the first drive-sleeve portion, and a trigger assembly operably associated with the housing and coupled to the first knife-sleeve portion,
the disposable portion of the surgical instrument including an end-effector assembly coupled to the distal end of the second shaft portion, a second knife-sleeve portion slideably disposed at least partially within the second shaft portion, and a second drive-sleeve portion slideably disposed at least partially within the second knife-sleeve portion;
positioning a coupling member within the second shaft portion for operably coupling the first knife-sleeve portion to the second knife-sleeve portion; and
connecting the first drive-sleeve portion to the second drive-sleeve portion.

16. The method of claim 15, wherein positioning the coupling member within the second shaft portion includes fixedly connecting a proximal end of the second knife-sleeve portion within an annular recess formed in the coupling member.

17. The method of claim 15, further comprising threadedly connecting the second shaft portion to the first shaft portion.
